(19) United States
(12) Patent Application Publication
(10) Pub. No.: US 2017/0156378 A1
(43) Pub. Date: Jun. 8, 2017

(54) METHOD AND APPARATUS FOR PURIFYING LIQUID

(71) Applicant: NESTEC S.A., Vevey (CH)

(72) Inventors: Celine Rimbault, Vittel (FR); Renaud Sublet, Vittel (FR)

(73) Assignee: NESTEC S.A., Vevey (CH)

(21) Appl. No.: 15/327,113

(22) PCT Filed: Jul. 9, 2015
(86) PCT No.: PCT/EP2015/065707

§ 371 Date: Jan. 18, 2017

(30) Foreign Application Priority Data
Jul. 18, 2014 (EP) 14177682.3

(51) Int. Cl. A23L 3/28; A23L 2/50; B67D 1/08; F25D 31/00; F28D 7/02; F28D 7/10; C02F 1/32; A61L 2/10

(52) U.S. Cl. CPC A23L 3/28; C02F 1/325; A23L 2/50; A61L 2/10; F25D 31/002; F28D 7/024; F28D 7/106; B67D 1/0859; C02F 2201/003; C02F 2201/3222; C02F 2201/3227; C02F 2303/04; A23V 2002/00; B67D 2210/00015

(57) ABSTRACT

An apparatus (100) for purifying liquid comprises an irradiation chamber (102) upon which are disposed a plurality of UV-LEDs (112) which irradiate a flow (110) of liquid as it passes therethrough; a coolant conduit (104) is disposed about said irradiation chamber (102) and UV-LEDs (112), which conducts a flow (126) of a coolant fluid through it thereby cooling the flow (110) of liquid and the plurality of UV-LEDs (112).

### TECHNICAL FIELD

The present invention relates generally to a liquid purification apparatus. More particularly, the present invention relates to providing a cooling means for an ultraviolet light-emitting diode irradiation system. The present invention also relates to a method for purifying a volume of liquid with such an apparatus, as well as a beverage dispenser comprising it.

### BACKGROUND OF THE INVENTION

The present invention relates generally to a liquid purification apparatus, and more particularly to providing a cooling means for an ultraviolet light-emitting diode irradiation system. It also relates to a method for purifying a volume of liquid with such an apparatus, and a beverage dispenser comprising it.

One of the most essential tasks in purifying liquids such as water for drinking is disinfection, so as to ensure that any pathogenic microorganisms (e.g. bacteria, viruses, and protozoans) present in the water cannot cause illness in anyone who drinks it. It is known to perform this disinfection by the process of ultraviolet (UV) irradiation, where a volume of water being treated is bombarded with high-energy radiation in the form of UV light. The UV light damages the DNA and RNA of the pathogenic microorganisms, destroying their ability to reproduce and effectively neutralizing their ability to cause disease.

Since such systems use light to disinfect, their effectiveness is reduced on liquid which is not naturally clear or which has not been filtered to remove suspended solids. The scope of “purification,” for the purposes of this document, should thus be understood as encompassing the disinfection of liquid in which turbidity is minimal.

Traditional UV liquid purification systems have employed gas-discharge lamps as UV sources, in particular mercury-vapor lamps. Recently, it has become more and more common to employ ultraviolet light-emitting diodes (UV-LEDs) as a source of ultraviolet light for irradiation. UV-LEDs have numerous advantageous aspects which makes them appealing for use in an ultraviolet liquid purification system, notably their compact size, robustness, energy efficiency, and lack of toxic components such as the mercury vapor found in conventional lamps. The solid-state nature of UV-LEDs also enables them to be switched on and off instantly, a further advantage relative to conventional gas-discharge lamps.

It should be noted that, in this document, the term “ultraviolet light-emitting diode” is abbreviated to “UV-LED,” and that any incidence of the latter term should not be interpreted otherwise.

While UV-LEDs do offer considerable advantages over traditional mercury-vapor lamps, their implementation does present other challenges. Despite their improved efficiency relative to mercury-vapor lamps, UV-LEDs emit a significant amount of heat during operation. This in turn causes the UV-LED to heat up, a condition exacerbated by the relatively high power-to-volume ratio of the UV-LED. At continued elevated temperatures, the optical power output and service lifetime of the UV-LEDs will be greatly diminished.

Prior art systems have attempted to address this, employing heat sinks to draw heat out of the UV-LEDs. The heat sinks are then themselves cooled by either natural convection or forced airflow. For example, the document KR2010-0027201 describes a system for cooling UV-LEDs in a water purification apparatus, wherein a fan directs air over a heat sink to which the UV-LEDs are connected.

Such configurations are disadvantageous, in that they require the heat sink to have a great deal of surface area to effectively dissipate all of the heat generated by the

UV-LEDs. Moreover, the amount of heat that can be dissipated is dependent on the air flow through the heat sink, the material from which it is fabricated, and the ambient temperature. A high-power UV-LED array, or one which is to be employed in an area of high ambient temperature, will require a very large heat sink and fan, increasing the cost to construct and operate the system and the noise generated during its operation.

It is therefore an object of this invention to resolve at least some of the foregoing difficulties of the prior art, or at least to provide a useful alternative.

### SUMMARY OF THE INVENTION

According, therefore, to a first aspect, the invention is directed towards an apparatus for purifying liquid, comprising a substantially tubular irradiation chamber adapted to conduct a flow of liquid therethrough, and a plurality of UV-LEDs disposed upon said irradiation chamber and adapted to irradiate said flow of liquid.

According to the invention, the apparatus comprises a coolant conduit disposed about said irradiation chamber and said UV-LEDs, said coolant conduit being adapted to circulate a flow of a coolant fluid about said irradiation chamber.

This is advantageous in that the waste heat generated by the UV-LEDs during their operation is dissipated, and their temperature during and after operation thereby controlled.

This is also advantageous in that the provision of the coolant conduit and the coolant fluid circulating therethrough will improve the efficiency with which the UV-LEDs are cooled. Specifically, the provision of the cooling conduit enables the provision of a coolant fluid which has a higher specific heat than that of the ambient air, thereby removing more heat from the irradiation chamber and the UV-LEDs for a given mass flow rate.

Moreover, the use of the coolant fluid to cool the irradiation chamber and UV-LEDs means that the cooling efficiency of the apparatus is independent of the ambient temperature and humidity.

In this way, the user can realize a reduction in the size of the apparatus, an increase in its effective power, or a combination of the two.

In one possible embodiment of the invention, the apparatus further comprises a first tube disposed coaxially about said irradiation chamber, and a second tube disposed coaxially about said first tube, said first and second tubes thereby defining between them a substantially annular space at least partially constituting the coolant conduit.

In this way, the total size of the irradiation chamber and the coolant conduit are minimized. This offers an increased flexibility in the application of a system incorporating an apparatus so configured.

In another possible embodiment of the invention, the coolant conduit is a tube at least partially configured as a helix having an axis substantially coincident with a longitudinal axis of the irradiation chamber.

This is advantageous in that the helical shape of the coolant conduit will maximize the volume of the coolant conduit that is disposed about the irradiation chamber, and thus maximize the amount of heat that the coolant fluid can absorb at any given flow rate. The cooling efficiency of the apparatus, and by extension the maximum number and intensity of the UV-LEDs, is thereby maximized.

Preferably, the coolant fluid is water.

This is advantageous in that water, having a high specific heat, will absorb a great deal of heat from the UV-LEDs during operation, reducing or even eliminating the need to chill the water coolant fluid to achieve sufficient cooling of the UV-LEDs.

Moreover, the use of water as the coolant fluid enables one to cool the apparatus with the liquid that is purified therein. This is particularly advantageous in systems such as water coolers which are generally provided with means for cooling the water, such that an apparatus according to the invention may be furnished a supply of chilled coolant water without necessitating any additional equipment, space, or expense.

Alternatively, the coolant fluid is a refrigerant gas.

In this way, the UV-LEDs are cooled to a lower temperature than can be achieved by circulating coolant fluid at ambient temperature.

Most preferably, the cooling conduit at least partially constitutes an evaporator of a refrigeration system.

This is particularly advantageous in that disposing the evaporator of a refrigeration system about the irradiation chamber in such a way will maximize the amount of heat transferred from the irradiation chamber and the UV-LEDs and, as a result, minimize the temperature to which they are cooled.

Furthermore, the evaporator will also maximize the degree to which the water within the evaporation chamber is cooled, thereby chilling the water as well as cooling the UV-LEDs and the irradiation chamber. The size and weight of the apparatus, as well as any beverage dispenser or similar device incorporating it, can be thereby reduced.

In a possible embodiment, the irradiation chamber and the coolant conduit define an interstitial space between them.

This is advantageous in that the interstitial space is ideally situated to accommodate the necessary electrical supply wiring for the UV-LEDs. The overall size of the apparatus is thereby minimized.

Most preferably, the interstitial space is at least partially filled with a heat-conducting material.

In this way, the efficiency with which the coolant fluid removes heat from the irradiation chamber and UV-LEDs is further improved.

In another possible embodiment, the cooling conduit is in fluid communication with a cavity of the irradiation chamber.

This is advantageous in that the water that is to be irradiated in the irradiation chamber also serves to cool the irradiation chamber and UV-LEDs. Therefore, any system employed to chill the water will also chill the irradiation chamber and UV-LEDs, minimizing the expense of implementing the apparatus in that no additional system for cooling a separate coolant fluid is necessary.

According to a second aspect, the invention is directed to a beverage dispenser comprising an apparatus for purifying liquid as heretofore described.

Such an apparatus is advantageous in that it will realize the advantages of the purification apparatus as described above.

According to a third aspect, the invention is directed to a method for the purification of a liquid, comprising the steps of providing a substantially tubular irradiation chamber adapted to conduct a flow of liquid therethrough, and a plurality of UV-LEDs disposed upon said irradiation chamber and adapted to irradiate said flow of liquid; providing a flow of a coolant fluid; directing said flow of a coolant fluid through a coolant conduit disposed about said irradiation chamber and said UV-LEDs, thereby cooling said irradiation chamber and said UV-LEDs; and directing a flow of liquid through said irradiation chamber, thereby irradiating said flow of liquid.

This is advantageous in that the performance of such a method will provide a purified liquid such as water to a user while realizing the advantages of a liquid purification system as described above.

In a preferred embodiment, the flow of coolant fluid is directed through the coolant conduit in a direction substantially opposite the direction of the flow of liquid through the irradiation chamber.

This is advantageous in that, as it establishes a cross-flow relationship between the liquid flowing through the irradiation chamber and the coolant fluid flowing through the conduit, the efficiency at which the irradiation chamber and the UV-LEDs are cooled is maximized.

Most preferably, the coolant fluid is water.

This is advantageous for the reasons enumerated above.

In another possible embodiment, the flow of coolant fluid directed through the coolant conduit is also the flow of liquid irradiated in the irradiation chamber.

In this way, the execution of the method is simplified, in that it avoids the need to provide separate loops for the coolant fluid and the liquid, as well as avoids any possible safety issues that may arise in the case of leaks or cross-contamination of the two flows. Furthermore, the direction of the liquid through the coolant conduit and irradiation chamber of the apparatus can be performed by implementing a simple plumbing connection, minimizing the cost of implementing the method.

Preferably, the flow of liquid is chilled prior to being directed through the coolant conduit and the irradiation chamber.

This is advantageous in that the temperature of the flow of liquid will not vary with the ambient conditions. This ensures that the cooling of the irradiation chamber and the UV-LEDs is always performed at a substantially constant efficiency.

In an alternate embodiment of the invention, the coolant fluid is a refrigerant gas, the coolant conduit thereby constitutes at least part of an evaporator of a refrigeration system, and the flow of liquid is cooled by said flow of refrigerant gas in said evaporator.

This is advantageous in that the configuration of the coolant conduit as an evaporator of a refrigeration system will maximize the cooling of the irradiation chamber and the UV-LEDs, and thereby the cooling capacity and efficiency of the system.

Moreover, it will also have the effect of chilling the liquid as it passes through the irradiation chamber. This is particularly advantageous when the method is embodied in a beverage dispensing device, as such devices are frequently configured to dispense chilled or refrigerated beverages.

Preferably, the flow of coolant fluid is provided at a temperature at or below 10° Celsius.

This is advantageous in that it will maximize the efficiency at which the irradiation chamber and UV-LEDs are cooled when the coolant fluid is directed through the coolant conduit.

### BRIEF DESCRIPTION OF THE FIGURES

FIG. 1 is a section view of an apparatus for purifying liquid according to a first embodiment;

FIG. 2 is a section view of an apparatus for purifying liquid according to a second embodiment; and

FIG. 3 is a side view of an apparatus 300 for purifying liquid, according to a third embodiment.

### DETAILED DESCRIPTION

For a complete understanding of the present invention and the advantages thereof, reference is made to the following detailed description of the invention.

It should be appreciated that various embodiments of the present invention can be combined with other embodiments of the invention and are merely illustrative of the specific ways to make and use the invention and do not limit the scope of the invention when taken into consideration with the claims and the following detailed description. In the present description, the following words are given a definition that should be taken into account when reading and interpreting the description, examples and claims.

In particular, the initialism “UV-LED” is employed for the sake of convenience and brevity to stand for “Ultraviolet Light-Emitting Diode,” and should not be construed as carrying any other meaning.

Furthermore, the term “irradiate” and variants thereof are to be understood in the context of sterilization processes by ultraviolet irradiation as described above, and as importing the technical characteristics of such processes.

Also, the term “refrigerant gas” should be understood as describing those substances which are employed as the working fluid in a refrigeration cycle; and which as a category are generally, but not necessarily, in a gaseous phase at standard temperature and pressure. Such substances need not necessarily be in the form of a gas at every phase of the refrigeration cycle, or in any particular phase of said cycle, but may in fact be present in the form of a gas, a liquid, or a combination of gas and liquid. The term “refrigerant gas” is thus a simplification for the sake of convenience.

Finally, as used in this specification, the words “comprises”, “comprising”, and similar words, are not to be interpreted in an exclusive or exhaustive sense. In other words, they are intended to mean “including, but not limited to.

Any reference to prior art documents in this specification is not to be considered an admission that such prior art is widely known or forms part of the common general knowledge in the field.

The invention is further described with reference to the following examples. It will be appreciated that the invention as claimed is not intended to be limited in any way by these examples.

The main principle of the invention is first described.

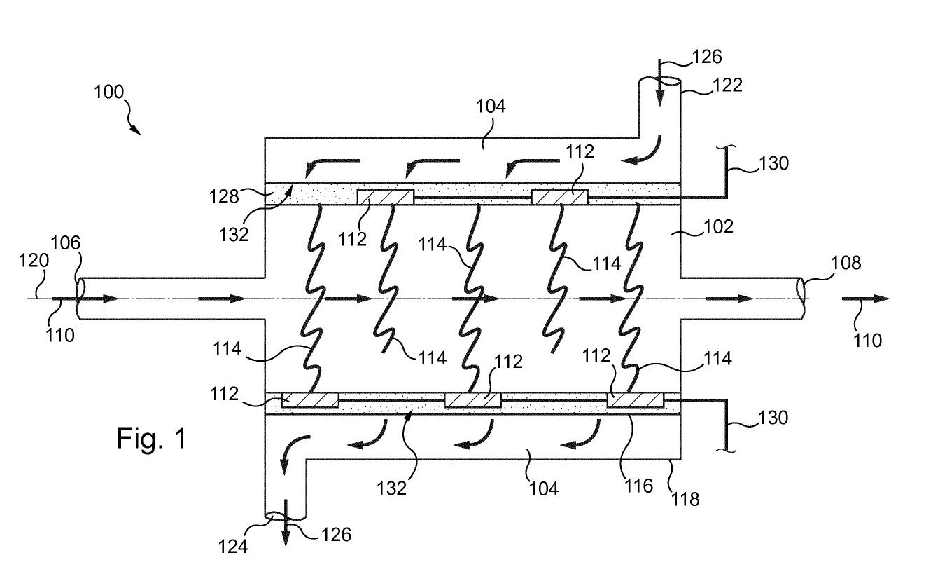

FIG. 1 is a section view of an apparatus 100 for purifying a liquid according to a first embodiment of the invention. The apparatus 100 comprises globally an irradiation chamber 102 and a coolant conduit 104, which will be discussed in turn.

The irradiation chamber 102 is provided with an irradiation chamber inlet 106 and an irradiation chamber outlet 108, such that a flow 110 of liquid is conducted through the irradiation chamber 102 in the manner depicted. About the perimeter of the irradiation chamber 102 are disposed a plurality of UV-LEDs 112. The UV-LEDs 112 are disposed so as to project UV light 114 into the irradiation chamber 102. In this way, the flow 110 of liquid is irradiated as it passes through the irradiation chamber 102, being thereby sterilized.

It will be readily recognized that the positioning of the UV-LEDs 112 as depicted here is simplified for considerations of clarity, and that in practice it may be preferable to adopt a different distribution thereof. It may, for instance, be preferable to dispose the UV-LEDs with a substantially uniform spacing along the length and around the circumference of the irradiation chamber, so as to more uniformly distribute the irradiation and heat emission of said UV-LEDs.

The coolant conduit 104 is formed by the tubular inner wall 116 which is disposed about the irradiation chamber 102, and the tubular outer wall 118 which is disposed about the inner wall 116. The irradiation chamber 102, and the inner wall 116 and outer wall 118 are all disposed substantially coaxially about the longitudinal axis 120.

The coolant conduit 104 is further provided with a coolant inlet 122 and a coolant outlet 124. When a flow 126 of a coolant fluid is introduced into the coolant inlet 122, it will circulate through the coolant conduit 104 about the irradiation chamber 102, and then out the coolant outlet 124. As the flow 126 of coolant circulates through the coolant conduit 104, it absorbs heat from the UV-LEDs 112 and the flow 110 of liquid.

The apparatus 100 may be configured so that the flow 126 of coolant runs in a direction counter to that of the flow 110 of liquid. Such a counter-flow arrangement will improve the cooling efficiency of the apparatus 100.

The inner wall 116 is not disposed flush against the irradiation chamber 102, but is instead slightly larger so as to accommodate the UV-LEDs 112. This results in an interstitial space 128 between the irradiation chamber 102 and the inner wall 116 of the coolant conduit 104.

The interstitial space 128 permits the passage of the electrical wires 130 to the UV-LEDs 112, thereby facilitating the supply of electricity to them. Since the electrical wires 130 do not penetrate the irradiation chamber 102 or the coolant conduit 104, the apparatus 100 is rendered essentially leak-proof. Moreover, maintenance of the UV-LEDs 112 and the electrical wiring 130 is facilitated; the user need only slide the inner wall 116 and the outer wall 118 of the coolant conduit 104 along the longitudinal axis 120 to expose them for servicing.

The interstitial space 128 may be left exposed to the atmosphere, or it may preferably be filled with a heat-conducting material 132. The heat-conducting material 132 may be thermal grease or paste, gel, cream, putty, or the like. When packed into the interstitial space 128, the heat-conducting material 132 will facilitate the conduction of heat from the flow 110 of liquid, the irradiation chamber 102, and the UV-LEDs 112 into the flow 126 of coolant within the coolant conduit 104. The inner wall 116 may also be configured such that it is in contact with the UV-LEDs 112.

Of course, the person of skill in the art will readily recognize how the precise configuration and dimensions of the irradiation chamber and the coolant conduit can be adapted to the needs of any particular application.

In particular, the volume of the irradiation chamber 102 and of the coolant conduit 104 is preferably, though not necessarily, adapted to the flow rate of the flow 110 of liquid through the apparatus 100. In addition, as the heat transfer rate from the irradiation chamber 102 and UV-LEDs 112 to the flow 126 of coolant is dependent at least in part on the area of interface between these components, the volume of the coolant conduit 104 should be no larger than is necessary to provide a sufficient mass flow rate of the flow 126 of coolant through the apparatus 100.

In a practical embodiment, the apparatus 100 could be integrated into a beverage dispensing apparatus. Such a dispensing apparatus could be simply a water fountain, or a machine for preparing food or drink such as soup or coffee. Such an apparatus could comprise, in addition to the apparatus 100, chillers or refrigeration units, storage tanks, pumps, power supplies, boilers and/or vaporizers, dispensers, and any other such material as would be necessary or desirable for integration into a beverage dispensing unit. Beverage dispensing apparatuses are generally well known in the art, and as such are not discussed further.

As a result, the dimensions and form of the irradiation chamber may vary according to the application in which it is to be employed. For instance, a point-of-use drinking water dispenser might have an irradiation chamber volume of approximately 100 cm3, with a flow rate of 1.5 to 2 liters per minute, while a single-serving hot beverage dispenser such as a domestic coffee maker or infant formula dispenser might utilize an irradiation chamber having a flow rate between 0.3 and 0.4 liters per minute. A vending machine, commercial coffee maker, or other such unit that might be found in commercial service might require a larger irradiation chamber to accommodate a higher flow rate and/or pressure, and possibly to achieve a greater degree of irradiation in the liquid. One such embodiment may have an irradiation chamber around 600 cm3 and a flow rate of about 2 liters per minute.

Of course, it will be readily recognized that characteristics such as the size and shape of the irradiation chamber; the number, position, and intensity of the UV-LEDs; and the temperature, flow rate, and pressure of the liquid and coolant fluid must be adapted to the particular application in question. Many different, alternative embodiments can thus be conceived, of which two will now be discussed.

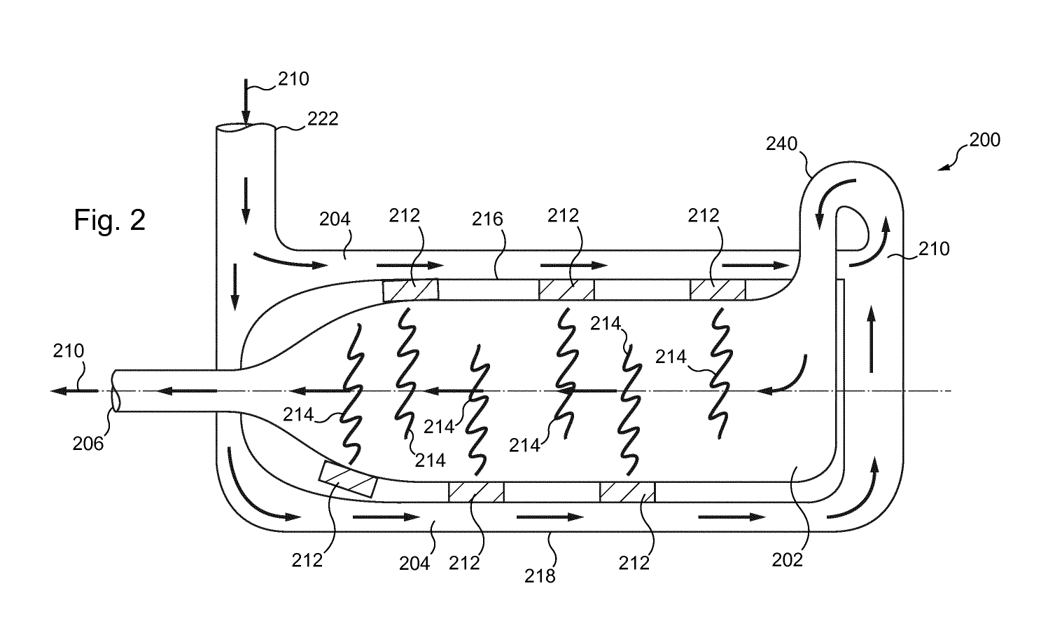

FIG. 2 is a section view of an apparatus 200 for purifying liquid according to a second embodiment.

The apparatus 200 is similar to the apparatus 100 depicted in and described with relation to FIG. 1, in that it comprises an irradiation chamber 202 and a coolant conduit 204, the latter being formed from the inner wall 216 and the outer wall 218. There are disposed upon the irradiation chamber 202 a plurality of UV-LEDs 212, which irradiate a flow 210 of liquid.

The flow 210 of liquid is first directed into the inlet 222. The inlet 222 feeds the coolant conduit 204, such that the flow 210 of liquid passes by the UV-LEDs 212, cooling them. The flow 210 then passes through the u-tube 240, which directs the flow 210 into the irradiation chamber 202. The flow 210 is then irradiated with UV radiation 214, and finally exits the apparatus 200 through the outlet 206.

In this way, the flow 210 serves as the coolant fluid even as it is, itself, irradiated. Such an arrangement is particularly advantageous where the flow 210 of liquid is provided in a chilled state, or where the flow 210 of liquid is cooled to the required temperature by means external to the apparatus 200. In either case, it is preferable that the flow 210 of liquid be at a temperature no greater than 10° Celsius. This ensures both the effective cooling of the UV-LEDs 212 and that the resulting liquid is at a temperature that is pleasant and refreshing to drink.

Moreover, the high specific heat of water means that, when employed as the coolant, its temperature will not rise more than a few degrees after being passed through the coolant conduit 204 and cooling the UV-LEDs 212.

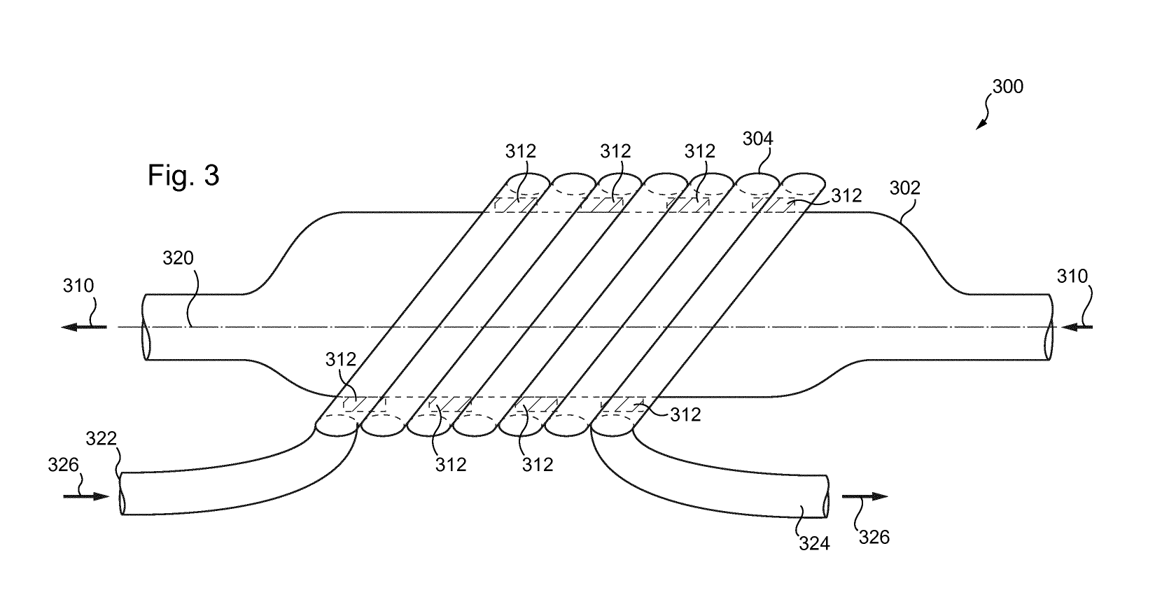

FIG. 3 is a side view of an apparatus 300 for purifying liquid, according to a third embodiment. The apparatus 300 comprises, as in the two embodiments previously presented, an irradiation chamber 302 through which a flow 310 of liquid is conducted, and upon which the UV-LEDs 312 are disposed. As the flow 310 of liquid passes through the irradiation chamber 302, the UV-LEDs 312 irradiate it. As in the previous embodiments, there may be a thermally-conductive material in a space between the irradiation chamber and the coolant conduit 304, here omitted for clarity.

The apparatus 300 further comprises the coolant conduit 304. The coolant conduit 304 is in the form of a helical coil of tube having an axis coincident with the longitudinal axis 320 of the irradiation chamber 302. In this embodiment, the coolant conduit 304 constitutes the evaporator coil of a refrigeration system; as such, the inlet 322 receives a flow 326 of a refrigerant gas from an expansion valve, which passes through the coolant conduit 304 before exiting by the outlet 324 to a compressor of said refrigeration system. The refrigerant gas is preferably selected from R-134a, R-410a, or R-600, as these refrigerants are among the most commonly used for domestic and commercial refrigeration and their characteristics are well known.

Of course, the coolant conduit 304 does not necessarily constitute the whole of the evaporator; indeed, in it may be that the helical coolant conduit 304 only represents a portion of the evaporator, and that the remainder thereof is disposed elsewhere or employed to realize a different effect, e.g. maintaining the temperature of liquid that has already been purified.

The precise dimensional and operative characteristics of the refrigeration system will therefore depend on the particularities of the application in which it is used. The person of ordinary skill in the art will be capable of adapting characteristics such as evaporator coil size, shape, and composition; refrigerant type, pressure, and charge weight, and so on.

Thus, as the flow 326 of coolant evaporates in the coolant conduit 304, it will chill both the UV-LEDs and the flow 310 of liquid through the irradiation chamber. In certain embodiments, this may be employed to chill the flow 310 of liquid to the desired temperature for consumption.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

In addition, elements described in the foregoing disclosure should not be taken as being limited to the combinations and configurations described in the foregoing example embodiments. Recombination of the elements described above according to the particulars of each application should be considered as envisioned when not in direct contradiction to this disclosure.

Furthermore, it should be understood that the forms and configurations of the irradiation chamber and the coolant conduit as described in and with reference to the Figures are purely exemplary. In particular, it should be understood that a system employing a refrigerant gas as a coolant need not necessarily have its coolant conduit configured as a helical tube, nor must a system employing water as a coolant have its coolant conduit configured as a tube coaxial to the irradiation chamber.

Different forms or combinations of forms of the irradiation chamber and the coolant conduit may be employed, whether the coolant fluid is a refrigerant gas, water, or some other fluid substance. The configuration of the irradiation chamber and coolant conduit can thus be tailored for each application to realize optimal irradiation and cooling performance.

Also, while it is envisioned that an apparatus according to the present invention be integrated into a beverage dispensing apparatus, it may equally be possible to employ such an apparatus in other applications, for example in commercial, industrial, medical, or other such applications where reliable purification of a liquid is sought. In particular, it may be advantageous to incorporate such an apparatus into devices such as beverage vending machines, coffee or tea dispensers, or dispensers for prepared food such as soups, cereals, infant formula, or the like.

It should thus be understood that various changes and modifications to the presently preferred embodiments described herein will be apparent to those skilled in the art. Such changes and modifications can be made without departing from the spirit and scope of the present invention and without diminishing its attendant advantages. It is therefore intended that the appended claims be considered as including any embodiment which is derived at least partially from it.

1. An apparatus for purifying a liquid, comprising a substantially tubular irradiation chamber adapted to conduct a flow of liquid therethrough, and a plurality of UV-LEDs disposed upon the irradiation chamber and adapted to irradiate said-the flow of liquid, the apparatus comprises a coolant conduit disposed about the irradiation chamber and the UV-LEDs, the coolant conduit being adapted to circulate a flow of a coolant fluid about the irradiation chamber.

2. The apparatus of claim 1, comprising a first tube disposed coaxially about the irradiation chamber, and a second tube disposed coaxially about the first tube, the first and second tubes thereby defining between them a substantially annular space at least partially constituting the coolant conduit.

3. The apparatus of claim 1, wherein the coolant conduit is a tube at least partially configured as a helix having an axis substantially coincident with a longitudinal axis of the irradiation chamber.

4. The apparatus of claim 1, wherein the coolant fluid is water.

5. The apparatus of claim 1, wherein the coolant fluid is a refrigerant gas.

6. The apparatus of claim 5, wherein the cooling conduit at least partially constitutes an evaporator of a refrigeration system.

7. The apparatus of claim 1, wherein the irradiation chamber and the coolant conduit define an interstitial space between them.

8. The apparatus of claim 7, wherein the interstitial space is at least partially filled with a heat-conducting material.

9. The apparatus of claim 1, wherein the cooling conduit is in fluid communication with a cavity of the irradiation chamber.

10. A beverage dispenser comprising an apparatus for purifying liquid comprising a substantially tubular irradiation chamber adapted to conduct a flow of liquid therethrough, and a plurality of UV-LEDs disposed upon the irradiation chamber and adapted to irradiate the flow of liquid, the apparatus comprises a coolant conduit disposed about the irradiation chamber and the UV-LEDs, the coolant conduit being adapted to circulate a flow of a coolant fluid about the irradiation chamber.

11. A method for the purification of a liquid, comprising the steps of:
providing a substantially tubular irradiation chamber adapted to conduct a flow of liquid therethrough, and a plurality of UV-LEDs disposed upon the irradiation chamber and adapted to irradiate the flow of liquid;
providing a flow of a coolant fluid;
directing the flow of a coolant fluid through a coolant conduit disposed about the irradiation chamber and the UV-LEDs, thereby cooling the irradiation chamber and the UV-LEDs; and
directing a flow of liquid through the irradiation chamber, thereby irradiating the flow of liquid.

12. The method of claim 11, wherein the flow of coolant fluid is directed through the coolant conduit in a direction substantially opposite the direction of the flow of liquid through the irradiation chamber.

13. The method of claim 11, wherein the coolant fluid is water.

14. The method of claim 13, wherein the flow of coolant fluid directed through the coolant conduit is also the flow of liquid irradiated in the irradiation chamber.

15. The method of claim 14, wherein the flow of liquid is chilled prior to being directed through the coolant conduit and the irradiation chamber.

16. The method of claim 11, wherein the coolant fluid is a refrigerant gas, the coolant conduit thereby constituting at least part of an evaporator of a refrigeration system, and wherein the flow of water is cooled by the flow of refrigerant gas in the evaporator.

17. The method of claim 11, wherein the flow of coolant fluid is provided at a temperature at or below 10° Celsius.
